(19) United States
(12) Patent Application Publication
(10) Pub. No.: US 2004/0167397 A1
(43) Pub. Date: Aug. 26, 2004

(54) Medical system

(75) Inventors: Norbert Brill, Konstanz (DE); Jorg Freidinger, Bodman-Ludwigshafen (DE); Dick van Rijn, Hergatz (DE); Wilfried Senn, Kreuzlingen (CH); Manfred Baur, Kreuzlingen (CH)

Correspondence Address: HMTAG C/O STEFFEN STRAUSS, KREUZLIUGERSTR. 5, LEUGWIL, CH-8574, CH

(73) Assignee: HMT Holding AG, Lengwil (CH)

(21) Appl. No.: 10/372,260

(22) Filed: Feb. 25, 2003

(51) Int. Cl. A61B 5/05

(52) U.S. Cl. 600/427; 600/439

(57) ABSTRACT

A medical system for treatment with focussed acoustic waves having a locating device, a therapy device, a patient positioning device, power supply devices for these devices. The complete system is installed in an integrated manner and adjusted on a common equipment carrier. The carrier frame corresponds in width and length to the projected floor area of a standard hospital bed so that the complete system can be easily transported in its mounted and adjusted state.

### BACKGROUND OF THE INVENTION

The invention relates to a medical system according to the preamble of the patent application of the claim 1.

A medical system of this type is known from DE 197 46 956 C2. This known system serves the purpose of treating a patient with focussed acoustic waves. The patient is placed on a positioning device in the form of a table. That part of the patient's body to be treated, e.g. a concrement or a section of tissue is determined by means of a locating device. A therapy device generates acoustic waves which are focussed onto the determined part of the body by means of the locating device in order to there cause the desired therapeutical effect, e.g. to destroy a concrement or a tumour.

In this known system the locating device and the therapy device are each separate equipment which are each provided with their own power supply device. The locating device is designed as an X-ray unit with a C-or U-arm. In order that the system can be used, the locating device and the therapy device are mechanically coupled and mutually adjusted so that the focus of the acoustic waves generated by the therapy device falls into the isocentre of the X-ray locating device.

The locating device and the therapy device are relatively large and heavy equipment. The coupling and especially the mutual adjustment and electrical connection of the locating device and of the therapy device are troublesome, time-consuming and have to be carried out by specialist personnel. Transport of the system, whereby the individual components have to be separated from each other, is thus only carried out in exceptional cases.

### BRIEF SUMMARY OF THE INVENTION

The underlying idea of the invention is to create a medical system of the type mentioned above which is flexible with respect to its spatial application possibilities.

The essential thought of the invention is that that the locating device, the therapy device and the positioning device with their respective power supply devices are arranged and mounted in a common equipment carrier. The length and width of the system mounted in the equipment carrier are at the greatest as large as the projected floor area of a standard hospital bed, i.e. or only very slightly exceed these floor area dimensions.

The locating device, the therapy device and the positioning device are permanently installed in the equipment carrier and adjusted relative to each other. The resulting small floor area makes it possible to transport the system in its fully assembled state especially also in the treatment rooms of a clinic or a physician's practice. This results in the essential advantage that the system can be delivered and installed factory mounted and adjusted without the necessity of carrying out comprehensive mounting and adjustment tasks at the installation location. Furthermore, the system can be transported in the mounted state from one treatment location to another treatment location without the necessity of decoupling the individual devices which on the other hand would necessitate coupling and adjusting at the new installation location.

Therefore, the system can be taken in a comfortable manner from one room to another room in a building and it is also especially suitable for mobile application in which case the system can be rented out or made available to various users at various locations for a limited period of time.

The positioning device of the medical system according to the invention is expediently adjustable so that the patient lying on the positioning device with his or her body area to be treated can be positioned in the focus of the therapy device.

If the positioning device has been designed with a reclining chair on which the patient to be treated can sit then this reclining chair can be essentially included within the outside dimensions of the equipment carrier so that the simple transport possibility of the system is not influenced by the positioning device.

In an advantageous design the positioning device has a reclining table on which the patient can lie stretched out during the treatment. In order not to impair the transport possibilities of the system the design of the reclining table is such that it can be reduced in size preferably without the aid of tools. For this purpose, the reclining surface of the reclining table is expediently subdivided whereby the individual surface parts can be pushed together, folded together or folded up so that they no longer protrude beyond the outside perimeter of the equipment carrier.

Preferably, the reclining table of the positioning device is arranged on one of the narrow sides of the equipment carrier and transverse to its longitudinal extent. In this way, the reclining table is freely accessible from at least three sides so that a frail or handicapped patient can easily onto the reclining table or laid onto the reclining table with the aid of the nursing personnel. Moreover, the patient lying on the reclining table is easily accessible for the treating physician or for the treating personnel. The arrangement of the positioning device on an end narrow side of the equipment carrier also makes it possible that the reclining table can be provided with a large vertical lifting distance whereby especially lowering of the reclining surface of the reclining table is possible in order to make it easier to climb onto the reclining table. In this respect, the reclining table can be lowered so far, for example, such that the reclining surface has a climb-on height of less than 75 cm above the floor.

In order to obtain an as compact as possible arrangement of the system in the equipment carrier, the power supply devices for the therapy device, the locating device and the positioning device are arranged in the end area of the equipment carrier and the locating device and the therapy device are arranged on the narrow front end of the power supply device which is facing the positioning device. As a rule, the locating device is arranged such that it can be swivelled in order to spatially localise the body area to be treated and to position the locating device in the isocentre.

If the locating device is arranged at the narrow front end of the power supply device then the swivelling axis of the locating device runs preferably horizontal in the longitudinal direction of the equipment carrier. If the locating device is especially designed as an X-ray device with an X-ray source and an X-ray receiver on a swivable C-or U-arm, then this C-or U-arm is preferably arranged laterally bent at right angels. In this manner, the swivelling axis of the C-or U-arm and the shock wave source of the therapy device can lie above each other in vertical plane and the C-or U-arm is led laterally around the shock wave source.

The electrical power supply and control of the locating device, the therapy device and the positioning device are integrated into a compact unit in the equipment carrier. This has the advantage that the electrical components required can be considerably reduced so that a common cabling can be installed and electrical coordination of the locating device, therapy device and positioning device and their power supply devices is not necessary. Furthermore, the overall control of the locating device, the therapy device and the positioning device can be facilitated via a control and data bus system. The control and data bus system can be connected via one single interface with an operator and control desk, by means of which the locating device, the therapy device and the positioning device can be controlled and operated. The operator and control desk can be designed as a separate unit which can be connected cable-dependant or non-cable-dependant to the control and data bus system. The operator and control desk can also be integrated into the system and arranged on the equipment carrier.

The integration of the entire electrical and electronic components and especially their installation in a control and data bus system also facilitates remote diagnosis and/or maintenance of the entire system, e.g. via a GSM modem or a telephone modem. In this way, time saving and cost saving maintenance and servicing of the system is possible.

The locating device is expediently equipped with an imaging device, which is preferably provided with a monitor integrated into the operator and control desk so that the part of patient's body to be treated and the patient's positioning can be observed and assessed by the physician. Depending on the part of the body to be treated the patient can be placed in various ways on the positioning device.

Especially an alternative position of lying on the back or face down is possible and transposition of head and foot position of the patient is also possible e.g. for locating and treating the right or left kidney. In an advantageous design the image processing device is so designed that by entering the position of the patient mirror-imaging of the screen information can be made in such a way that the image on the monitor of the operator and control desk is always given in the same patient position, e.g. in such a way that the body of the patient is seen from the front and the head of the patient is at the top of the picture. The compensating mirror-imaging of the various positions of the patient on the positioning device can also be transferred to the control of the positioning device by means of the operator and control desk. In this way, the physician can carry out positioning on the operator and control desk if required in accordance with the mirrored images on the monitor screen whereby the positioning device is moved in a controlled manner corresponding to the actual position of the patient.

The complete medical system can be simply transported by means of the equipment carrier and installed stationary. If a change in the installation location is intended then it is preferable to design the equipment carrier to be transportable. In an advantageous design the equipment carrier has in this respect swivelling rollers or wheels at one end. The other end of the equipment carrier rests on the floor so that the system stands stable in its location. For transporting, a lifting truck is used which engages at the opposite end of the equipment carrier to the swivelling rollers or wheels in order to make the equipment carrier with the complete integrated system mobile. In this respect, the lifting truck engages preferably positively on the equipment carrier in order to lift it from the floor. The lifting truck is steerable in order to facilitate easy manoeuvring during moving. Expediently, the lifting truck can also be driven by a motor, e.g. by means of a battery-powered electric motor.

### BRIEF DESCRIPTION OF THE DRAWING

These and other features, aspects and advantages of the present invention will become better understood with reference to the following description, appended claims, and accompanying drawing, where:

FIG. 1 depicts a side view of the medical system of the first embodiment of the invention.

FIG. 2 depicts a different view of the system of FIG. 1.

FIG. 3 depicts a top view of the system of a second embodiment.

FIG. 4 depicts a side view of the system of a second embodiment during transportation.

FIG. 5 depicts a different view of FIG. 4.

FIG. 6 depicts a side view of the operating unit.

FIG. 7 depicts a different view of the operating unit.

FIG. 8 depicts a side view of the system of a third embodiment with integrated operating unit.

### LIST OF REFERENCE NUMBERS

10 equipment carrier

12 swivelling weels

14 feet

16 housing

18 X-ray source

20 X-ray receiver

22 C-or U-arm

24 swivelling axis

26 therapy head

28 coupling membrane

30 axis of the X-ray beam

32 positioning device

34 vertical lifting columns

36 horizontal guides

38 vertical guides

40 pneumatic unit

42 recess

44 surface parts

46 pneumatic units

48 lifting truck

50 swivelling wheels

52 handle

54 operator and control desk

56 operator's panel

58 imaging device

Equivalent parts are indicated by the same reference numbers.

### DETAILED DESCRIPTION OF THE INVENTION

The invention summarized above and defined by the enumerated claims may be better understood by referring to the following detailed description, which should be read in conjunction with the accompanying drawing. This detailed description of a particular preferred embodiment, set out below to enable one to practice the invention, is not intended to limit the enumerated claims, but to serve as a particular example thereof.

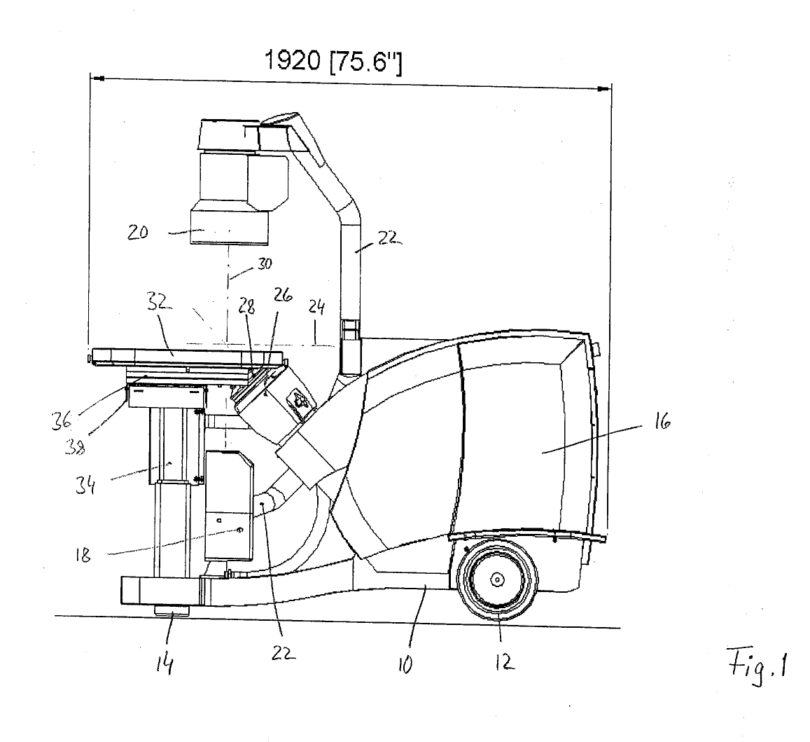

FIG. 1 shows the medical system in a first embodiment.

An equipment carrier 10 carries the complete system. The equipment carrier 10 has essentially the shape of a horizontal rectangle and is formed by a simple, one-piece light steel structure. The equipment carrier 10 has swivelling wheels 12 at its narrow end. At the other narrow end the equipment carrier 10 stands on the floor with feet 14.

On the equipment carrier 10 is located a housing 16, which takes up about half of the longitudinal extent of the equipment carriers. At the front narrow end of the housing 16 there is a device for locating and/or diagnosis (brief locating device) of the body part of a patient to be treated, a device for generating and focusing acoustic waves (brief therapy device) onto the body area to be treated and a device for accommodating the patient to be treated (brief positioning device).

The locating device represented in the design example is an X-ray locating device consisting of an X-ray source 18 and an X-ray receiver 20. The X-ray source 18 and the X-ray receiver 20 are fitted to the two free ends of a C-or U-arm 22. The C-or U-arm 22 is bearing-mounted in its central area onto the housing 16 and is swivable around a swivelling axis 24. The swivelling axis 24 runs horizontal and in the direction of the longitudinal axis of the equipment carrier 10. The mode of operation of such an X-ray locating unit is known state of the art technology.

Instead of an X-ray locating device as shown in the drawing another known locating device can also be used e.g. an ultrasound locating device.

The therapy device has a therapy head 26. In the therapy head 26 acoustic pressure waves for example are generated by means of an electric spark discharge in a liquid which are focused e.g. by means of a parabolic reflector as shock waves. The therapy head 26 is closed off by means of a coupling membrane 28 which is placed on the body of the patient in order to transmit the acoustic shock waves generated in the therapy head 26 into the body of the patient.

The axis 30 of the X-ray beam sent from the X-ray source 18 to the X-ray receiver 20 intersects the swivelling axis 24 of the C-or U-arm in an isocentre. This isocentre thus remains spatially fixed when the C-or U-arm 22 is swivelled around the swivelling axis 24. The therapy head 26 is arranged rigid on the housing 16 and aligned in such a way that its centre axis intersects the isocentre and the focus of the acoustic shock waves falls into this isocentre.

The positioning of the C-or U-arm 22 with the swivelling axis 24 lies in a vertical plane which runs through the longitudinal axis of the equipment carriers 10 above the therapy head 26. That is why the C-or U-arm 22 is laterally bent at right angles between the swivelling axis 24 and the X-ray source 18 and led in a bend around the therapy head 26.

The housing 16 accommodates the electrical supply and the electronic control circuit of the locating device and the therapy device. Likewise, if needed the housing 16 can also contain a preparing device for the liquid medium of the therapy head 26. The electronic circuits and controls of the power supply devices are combined via a common control and data bus system.

The positioning device has a horizontal reclining table 32 upon which the patient to be treated is positioned. The height of the reclining table 32 is adjustable by means of telescopic retractable vertical lifting columns 34. Moreover, the reclining table 32 can be moved towards and from the therapy device by means of horizontal guides 36. Further to the horizontal guides 36 there are vertical guides 38 which serve the purpose of moving the reclining table 32 transverse to the longitudinal direction of the frame of the equipment carrier 10. The height adjustment by means of the lifting columns 34 and the horizontal adjustment in the guides 36 and 38 is carried out preferably by means of a pneumatic or hydraulic or electric drive.

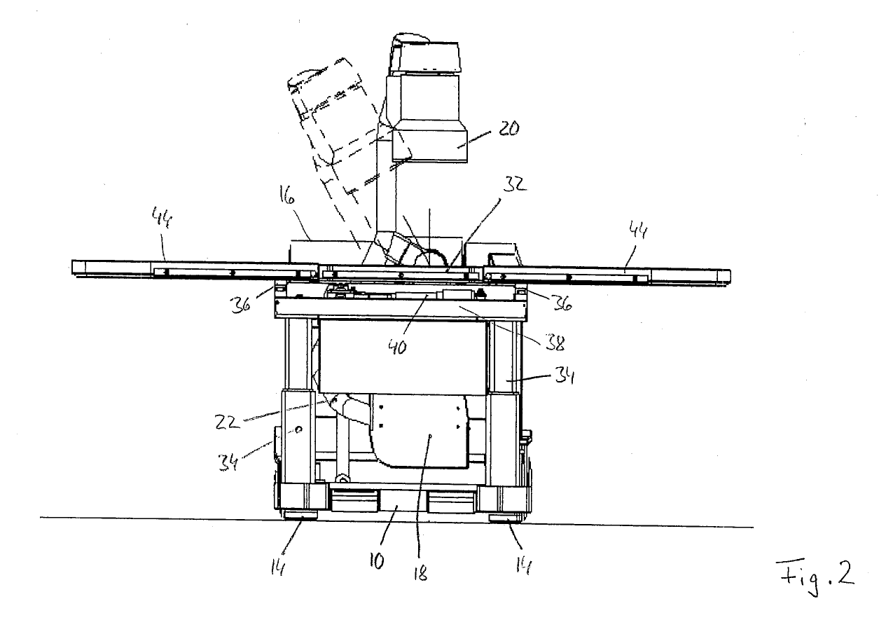

A pneumatic unit 40 for the horizontal transverse movement in the guides 38 can be seen in FIG. 2.

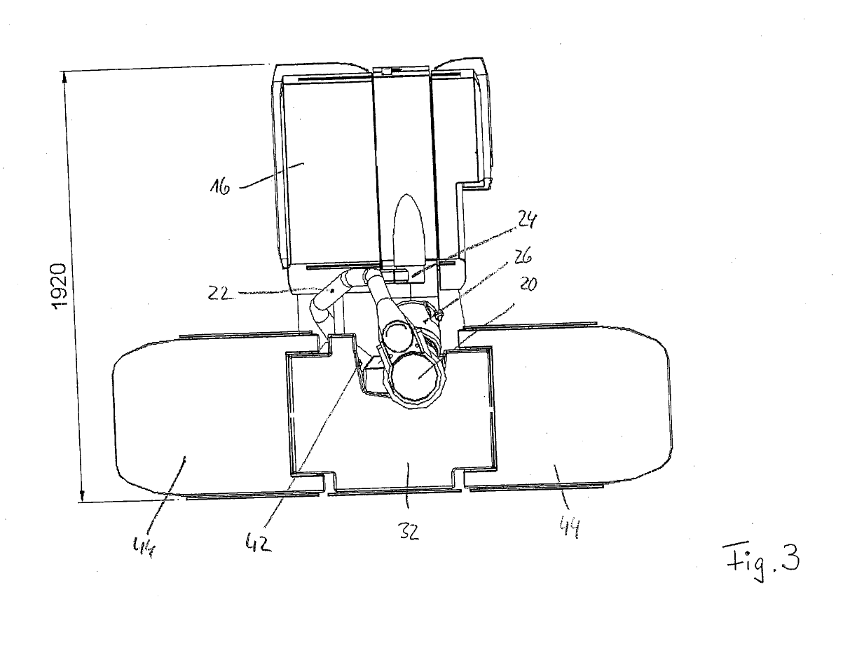

In middle area on the edge facing the housing 16 the reclining table 32 has a recess 42 as shown in FIG. 3. The patient is positioned on the reclining table 32 in such a manner that the part of the body to be treated e.g. a body concrement to be destroyed, a tumour, a bone area to be treated or similar lies on this recess 42. By means of the lifting columns 34 and the guides 36 and 38 the reclining table 32 with patient can be moved under control in the three spatial coordinates in order to position the part of the body to be treated as localised by the locating device in the isocentre and thus in the focus of the therapy head 26. In this respect, the signals of the locating device, e.g. the signals of the X-ray receiver 20 are fed to an image processor and represented in an imaging system e.g. on a monitor. By swivelling the C-or U-arm 22, observation under various spatial directions is possible in order to determine the spatial position of the body part to be treated.

In FIG. 2 is shown by means of a dotted line how the X-ray locating device can be swivelled from the vertical position by 30° in order to locate and localise the part of the body to be treated from two spatial directions.

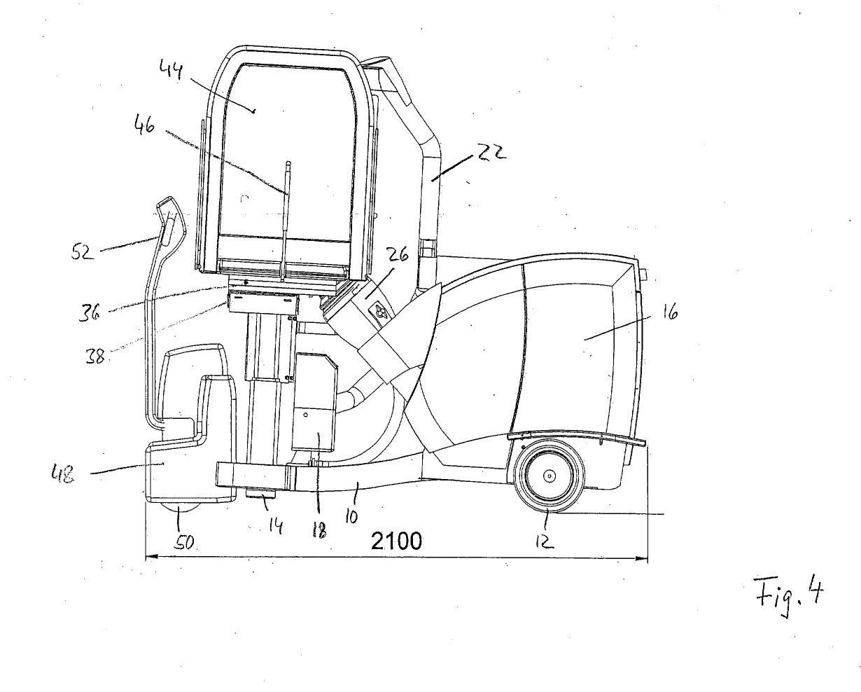

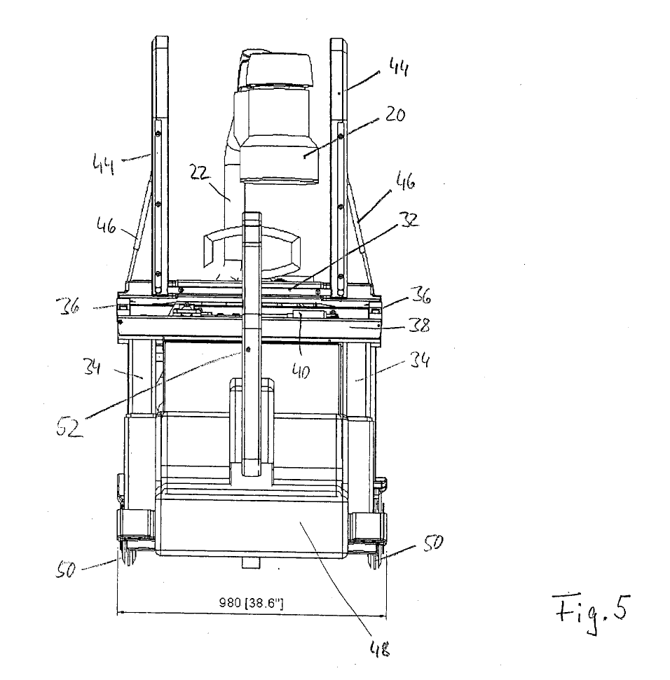

In FIGS. 2 and 3 is shown the reclining surface of the reclining table 32 divided into three surface parts. The middle surface part with the recess 42 is carried movable for positioning the patient in the guides 36 and 38. The two end parts 44 of the reclining surface protruding out to both sides beyond the equipment carrier 10 can be swivelled upwards around a horizontal axis running in the longitudinal direction of the equipment carrier 10 until they stand vertically upwards and are thus located within the floor area of the equipment carrier 10. Pneumatic units 46 are also used to swivel the end parts 44 upwards. As the end parts 44 in the FIGS. 1, 2 and 3 are shown folded down so that the reclining table 32 has a continuous horizontal reclining surface, the FIGS. 4 and 5 show the end parts 44 in the swivelled up position.

The length and width of the equipment system correspond to the floor area of a standard hospital bed or are possibly also smaller than this floor area. Correspondingly, the overall length of the integrated system arranged on the equipment carrier 10 is given with 1920 mm in FIGS. 1 and 3 and in FIG. 5 the width of the system with end parts 44 swivelled up is given with 980 mm.

In the design shown in the drawing in which the equipment carrier 10 is provided with swivelling wheels 12, the system is transportable. As represented in FIGS. 4 and 5 a lifting truck 48 can be used for this purpose and which is applied at the narrow end of the equipment carrier 10 at the opposite end to the swivelling wheels and which runs under the equipment carrier 10 in order to lift the equipment carrier 10 so that the feet 14 lift free from the floor. Preferably, the lifting truck 48 engages positively on the underside of the equipment carriers 10. The lifting truck 48 is provided with swivelling wheels 50 and preferably motor driven e.g. by means of a battery-powered electric motor. A handle 52 serves to guide and manoeuvre the transportable system on the swivelling wheels 12 of the equipment carrier 10 and the running wheels 15 of the lifting truck 48. Due to the above-mentioned dimensions of the equipment carriers 10 and of the integrated system arranged on the equipment carrier moving and manoeuvring of the system is also possible inside buildings and through door openings.

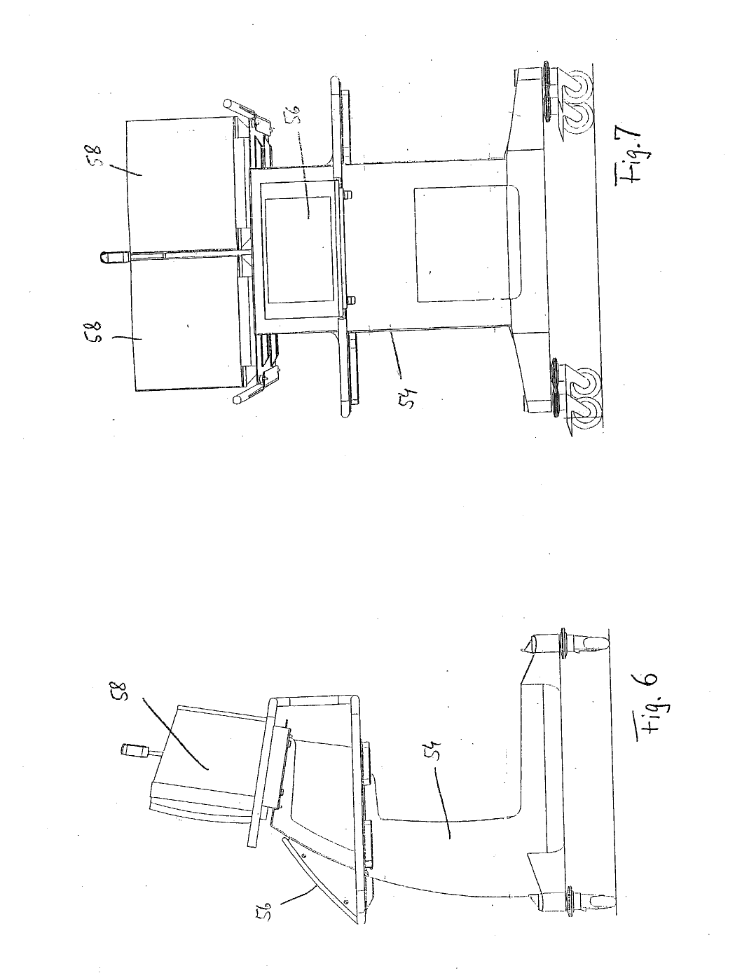

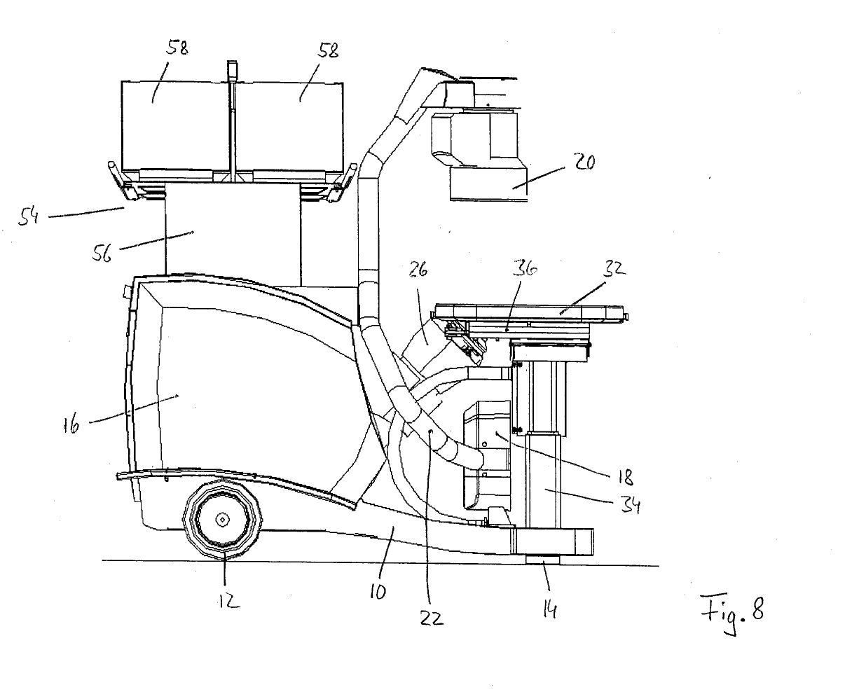

In the designs shown in FIGS. 1 to 5 the system is operated with a separate operator and control desk 54 which is shown in FIGS. 6 and 7. The operator and control desk 54 is designed as an independent transportable unit. The operator and control desk 54 has an operator's panel 56 which can be implemented for example as a touch screen. The operator's panel 56 contains switching elements for the operation and control of the locating device and of the therapy device as well as switching elements for moving the reclining table 32 in the three spatial axes. Furthermore, the operator and control desk 54 has display devices, e.g. monitors 58, in order to display the images received from the locating device. The operator and control desk 54 is connected to the system via cable or via a non-cable-dependent signal exchange.

FIG. 8 shows a further variation of the system in which the operator and control desk 54 is arranged on the equipment carrier 10 and is integrated into the system. For this purpose, the operator and control desk 54 is mounted on the housing 16 which results in a further space saving and ease of transport.

What is claimed is:

1. Medical system comprising
a locating device, means for locating and diagnosing a part of the body of a patient to be treated,
a therapy device, means for generation of acoustic waves and for focussing these waves into the body region to be treated,
a positioning device, means for positioning the patient to be treated,
power supply devices, means for power supply and control of said locating device, said therapy device and said positioning device, and
said locating device, said therapy device, said positioning device and said power supply devices being accommodated in a common equipment carrier, whereby said locating device and said therapy device being adjusted relative to each other in said common equipment carrier and
the projected floor area of said locating device, said therapy device, said positioning device, and said power supply devices is not exceeding in length and width the projected floor area of a standard hospital bed at least not considerably.

2. Medical system according to claim 1,
wherein said positioning device is positionable in relation to said locating device and said therapy device.

3. Medical system according to claims 1,
wherein said equipment carrier being designed a transportable equipment carrier.

4. Medical system according to claim 3,
wherein said transportable equipment carrier having swivelling wheels at one end and resting on the floor at its opposite end and by a lifting truck being provided with which this other end of the equipment carrier can be lifted to make the equipment carrier transportable.

5. Medical system according to claim 1,
wherein said positioning device having a reclining table protruding beyond the equipment carrier in the operating position in order to accommodate a patient, and which can be put into a transport position located within the perimeter of the equipment carriers.

6. Medical system according to claim 5,
wherein said reclining table being divided into at least two surface parts, with the surface parts which protrude beyond the equipment carrier being insertable, foldable or swivable upwards so that they do not protrude beyond the equipment carrier in the transport position.

7. Medical system according to claim 1,
wherein said positioning device being designed preferably as a reclining chair.

8. Medical system according to according to claim 1,
wherein said power supply devices of said locating device, said therapy device and said positioning device being integrated in a common housing and being electrically interconnected.

9. Medical system according to claim 8,
wherein the control and monitoring of said locating device and said therapy device being effected via a control and data bus system.

10. Medical system according to claim 9,
wherein said control and data bus system being connectable for remote diagnosis and/or remote maintenance.

11. Medical system according to claim 8,
wherein said locating device, said therapy device and said positioning device being monitored and controlled via a connected operator and control desk.

12. Medical system according to claim 11,
wherein said operator and control desk having an imaging device for displaying the image signals received from the locating device.

13. Medical system according to claim 12,
wherein the image signals of said locating device being fed to said imaging device via an image processor which carries out one or more image rotations which are aligned according to a patient position which can be entered.

14. Medical system according to claim 1,
wherein said locating device being an X-ray locating device with an X-ray source and an X-ray receiver with common axis being arranged on a C- or U-arm

15. Medical system according to claim 14,
wherein said C- or U-arm of said locating device is designed to be reducible in height.

16. Medical system according to claim 14,
wherein said C- or U-arm of said locating device is designed preferably foldable.

17. Medical system according to claim 14,
wherein said C- or U-arm being positioned swivable around an axis through the focussing point of said therapy device.

18. Medical system according to claim 14,
wherein said swiveling axis lies horizontally in the longitudinal centre plane of said therapy device.

19. Medical system according to claim 14,
wherein said C-or U-arm being bent at several angles beneath its swivable bearing axis and being led around said therapy head.
